(19) United States
(12) Patent Application Publication
(10) Pub. No.: US 2015/0257827 A1
(43) Pub. Date: Sep. 17, 2015

(54) LASER BEAM ALIGNING UNIT AND LASER TREATMENT DEVICE FOR TREATING A MATERIAL

(71) Applicant: Anton Kasenbacher, Traunstein (DE)

(72) Inventor: Anton Kasenbacher, Traunstein (DE)

(21) Appl. No.: 14/723,747

(22) Filed: May 28, 2015

Related U.S. Application Data
(62) Division of application No. 13/416,345, filed on Mar. 9, 2012.
Application No. 13/416,345 is a continuation of application No. PCT/DE2010/075079, filed on Aug. 18, 2010.

(30) Foreign Application Priority Data
Sep. 18, 2009 (DE) 10 2009 042 199.8

(51) Int. Cl. A61B 18/20

(52) U.S. Cl. CPC A61B 18/20; A61B 2018/2035; A61B 2018/2085; A61B 2018/2005

(57) ABSTRACT

The disclosure relates to a laser beam aligning unit comprising an outer sleeve and an inner sleeve arranged inside the outer sleeve such that a laser beam may be guided through an inner chamber thereof in a direction of an area of material to be treated. A laser treatment device may comprise the laser beam aligning unit and/or a distribution device for distributing an ectoine solution.

### RELATED APPLICATION

This application is a divisional of and claims priority to U.S. patent application Ser. No. 13/416,345, filed on Mar. 9, 2012, which is a continuation of and claims priority to PCT Application PCT/DE2010/075079, filed on Aug. 18, 2010, which claims priority to German Patent Application 10 2009 042 199.8, filed on Sep. 18, 2009. U.S. Patent Application 13/416,345, PCT Application PCT/DE2010/075079, and German Patent Application 10 2009 042 199.8 are incorporated herein by reference.

### FIELD

The present disclosure relates to a laser beam aligning unit for treating a material, a laser treatment device for treating a material, and/or a method for treating tissue.

### SUMMARY

The properties of laser radiation, (e.g., intensity and focusing ability) have resulted in lasers being used in fields related to material treatment. In laser-substrate interactions, a photon beam may be absorbed by a workpiece to be treated, and material may be excited, heated, vaporized, disassociated, and/or ionized as a function of energy from the photon beam. During laser treatment, the laser beam may be absorbed by a workpiece to be treated, and material of the workpiece may be heated, melted, and/or vaporized by high energy associated with the laser beam. In this way, holes may be drilled in metal plates and/or semiconductor wafers may be separated into individual semiconductor chips, for example. The laser treatment may generally be performed by way of pulsed laser radiation in special laser treatment devices. Typically, CO₂ and/or solid-state lasers, (e.g., such as Nd:YAG, Nd:YVO₄, and/or Nd:GdVO₄ lasers) may be used as laser beam sources. One aspect of a laser treatment may relate to throughput, (e.g., such as, for example, the number of holes which may be drilled into a metal plate within a unit of time). Throughput may be based on an undisturbed interaction of laser photons with the substrate, for example. Accordingly, there may be a demand for increasing efficiency of a laser while concurrently decreasing energy use associated therewith (e.g., reducing the number of costly photons).

It may be appreciated that material treatment may refer to materials of all types (e.g., materials of all physical states, natural or synthetic materials, inorganic or organic materials, and/or any combination thereof). One subset of materials may comprise biological tissue, (e.g., devital, vital biological tissue, and/or hard tissue, such as tooth material, for example). In one embodiment, aspects of the present disclosure may be applied to the field of dentistry, (e.g., instead of a mechanical drill for ablating and/or abrading tooth material, such as decayed tooth material, a laser treatment device may be used), for example. Additionally, other types of biological tissue and/or tissue types, (e.g., other hard tissue, soft tissue, and/or tissue liquids), may be affected. Another potential field of use may be the field of ophthalmology, for example.

Another aspect of this disclosure relates to promoting biosafety concurrent to treatment described herein, (e.g., avoiding effects of excessive ultraviolet radiation). A laser pulse may interact with a thin surface area of the material such that microplasma may be formed in a focus of the treatment laser beam. Such plasma (e.g., microplasma) may cause thermal damage (e.g., dehydration) and/or chemical damage (e.g., ultraviolet radiation). Stress factors related to the damage may be associated with health-damaging effects, for example. Therefore, it may be advantageous to avoid exogenous and/or endogenous noxious effects and/or circumstances while maintaining a higher ablation efficiency.

According to one aspect, the present disclosure may provide a laser beam aligning unit and/or a laser treatment device, configured to treat a material based on utilization of laser radiation delivered via a laser beam source (e.g., photon protection and/or photon reduction). According to another aspect, a method for treatment of a tissue may be provided, which may mitigate side effects associated with treatment, for example.

During treatment and/or ablation of materials using a laser beam, the ablated material, (e.g., even if suctioned away), may form an optical barrier to the laser beam, at least because ablated particles may be located (e.g., for an indeterminate time) in the beam path of the laser beam and thus act as an absorption and/or scattering center for the laser beam, for example. In one embodiment, an arrangement may be provided to enable the laser beam to reach a surface of the material to be treated in a substantially unobstructed manner.

According to one aspect, a laser beam aligning unit for treatment of a material is provided, comprising a first inner sleeve and a second outer sleeve, the inner sleeve arranged inside the outer sleeve such that a laser beam may be guided through an inner chamber, in a direction toward an area of material to be treated. For example, the laser beam aligning unit may be implemented as a handpiece and/or a part thereof, which may be a component of a laser treatment device comprising a laser beam source configured to provide a pulsed treatment laser beam.

The laser beam aligning unit may comprise an arrangement which enables focusing of the laser beam (e.g., in a near unobstructed manner on a surface of a material to be treated), for example. In one embodiment, the arrangement enables protection of laser photon from premature interactions with ablation products and/or air particles, for example. Ultimately, the arrangement may enable a laser beam source to be operated using less optical output power, thus conserving energy, for example.

According to one embodiment, the outer sleeve of the laser beam aligning unit may protrude beyond the light-outlet-side end of the inner sleeve (e.g., based on a predetermined distance in an axial direction of the respective sleeves). The predetermined distance may correspond to a Rayleigh length in relation to the focus of the laser beam bundle exiting from the light-outlet-side end of the inner sleeve. Additionally, the outer sleeve may be implemented such that the outer sleeve is supported on an outer sleeve edge on a surface of the material to be treated, such that the light-outlet-side edge of the inner sleeve may comprise a space based on a

Rayleigh length from the surface of the material to be treated. Focusing conditions may be provided using such an arrangement, for example. It may be appreciated that it may not be desirable for a protective glass attached on a light-outlet-side front side of the outer sleeve to be located within a focal length of the focused laser beam. To this end, the protective glass may be damaged by the laser beam as a result of residual absorption and/or ablating associated with multiphoton absorption. In one embodiment, the spacing between the light-outlet-side end of the inner sleeve and the edge of the outer sleeve may be selected as greater than the Rayleigh length, to mitigate damage to the protective glass by the laser, for example. According to another aspect, the spacing between the light-outlet-side ends of the sleeves and/or the spacing between the protective glass and the surface to be treated may be determined based on a risk level associated with soiling the protective glass by ablated material. Additionally, a cleaning mechanism may be provided, as will be discussed in greater detail below.

According to one embodiment, a first sleeve and/or a second sleeve may comprise a cylindrical and/or conical shape and comprise a diameter which decreases continuously in a direction toward a light-outlet-side end, for example.

According to another embodiment, a scanning unit may be provided and configured to scan an area based on the laser beam, scanning unit, guide optic for the laser beam, and/or dimensions of the inner sleeve (e.g., inner sleeve dimensions may be adapted such that a scanned area rests inside the inner sleeve as the laser beam passes through the inner sleeve, for example). The diameter and/or cross-section of the inner sleeve may thus be restricted such that the diameter may not become smaller than a desired scanning area of the laser beam, for example. Conversely, the scanning unit may be controlled such that the scanning area of the laser beam may be located inside the inner sleeve.

According to one embodiment, at least one supply and/or suction line may be integrated in a wall of the outer sleeve and/or in a wall of the inner sleeve. For example, the supply and/or suction line may extend in a longitudinal direction of the sleeve up to a respective light-outlet-side end, such that an end of the line may be (e.g., directly) opposite to a surface area to be processed. The line may be connected to a nozzle at this end, and maybe controlled and/or aligned remotely, for example. In one embodiment, one or more supply lines may be integrated in (e.g., comprised by) the inner sleeve and a suction line may be integrated in the outer sleeve. The supply lines may be designed to supply various media, (e.g., such as air, water, special media such as pharmaceutical substances, various types of organic or inorganic materials, free electrons, and/or cold plasmas), for example.

During treatment of tissue using pulsed laser radiation, harmful thermal and/or chemical stress may occur, (e.g., during a tooth treatment), and a suitable pharmaceutical medium may be applied (e.g., to an area of a tooth surface to be processed, as a precaution). Additionally, ectoine and/or derivatives thereof may form a suitable safeguard from noxious events and/or circumstances associated with laser treatment, for example.

According to one aspect, a laser treatment device for the treatment of tissue may be provided and configured to emit a pulsed treatment laser beam, for example. According to another aspect, a distribution device may be configured to distribute an ectoine solution, for example.

In one embodiment, a method for the treatment of tissue provides that a pulsed laser beam may be applied to an area to be treated to irradiate the area, and an ectoine solution may be applied to medicate tissue, for example. According to one embodiment, the ectoine solution may comprise an isotonic ectoine solution (e.g., comprising a same osmotic pressure as human blood). For example, an indispensable osmolality value may be determined to be 78 mosm/kg. To this end, isotonic formulas may be generated based on a concentration range from 0≦n≦3.588087% ectoine. According to one embodiment, 0.646682% NaCl may be added to a 1% ectoine solution, for example. According to one embodiment, the ectoine solution may comprise an aerosol form. Further, aerosol particles may range in sizes between 0.01 μm and 2 μm, for example.

According to one embodiment, a laser treatment device may be used to perform a corresponding method. The laser treatment device may be connected to a distribution device configured to distribute ectoine solution, for example. Additionally, feedthrough lines may be provided to supply ectoine solution and/or apply the ectoine solution to a target area of tissue to be treated (e.g., via an adjustable nozzle, which may be controlled and/or aligned, for example).

### DESCRIPTION OF THE DRAWINGS The disclosure is explained in greater detail hereafter in the form of further embodiments on the basis of the drawings. In the figures:

FIG. 1 illustrates a schematic view of a laser beam aligning unit in a longitudinal section according to one embodiment;

FIGS. 2A, 2B illustrates the aligning unit of FIG. 1 in a cross-section along line A-A (A) and in a cross-section along line B-B (B); and

FIG. 3 illustrates a schematic view of a laser treatment device according to one embodiment.

### DETAILED DESCRIPTION

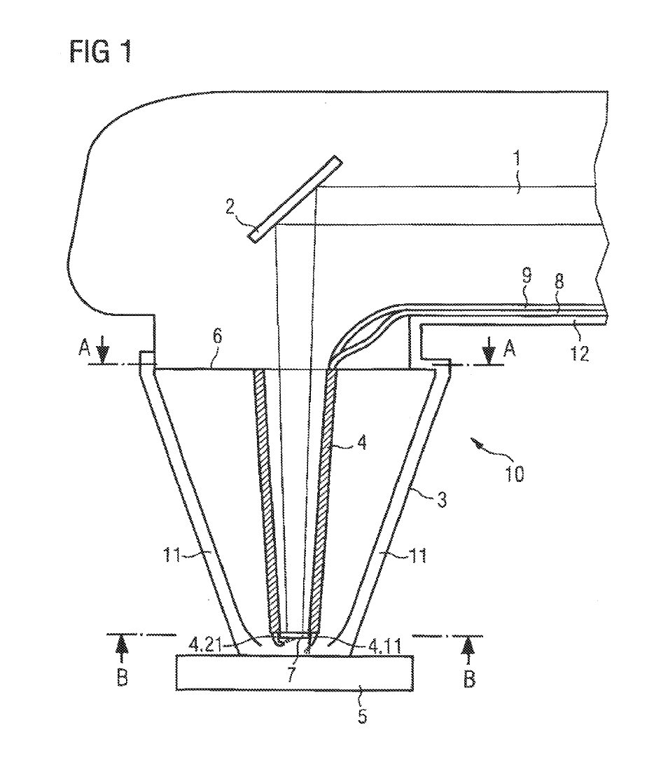

A laser beam aligning unit is illustrated in a longitudinal section according to one embodiment in FIG. 1. The laser beam aligning unit 10 may be utilized for aligning a treatment laser beam 1 on a workpiece 5, for example. The laser beam aligning unit 10 may comprise a horizontal section aligned parallel to the surface of the workpiece 5 (e.g., of which merely a portion is illustrated in the top area of FIG. 1). The horizontal section may be adjoined by a vertical section, and aligned vertically in relation to the surface of the workpiece 5, for example. The treatment laser beam 1 (e.g., coming from a laser beam source not shown) may be guided through the horizontal section incident therein on a deflection unit 2, and may be guided into the vertical section, and may be incident below the vertical section on the surface of the workpiece 5. The horizontal section may comprise focusing components (not shown) configured to focus the laser beam 1, (e.g., an objective and/or a lens), by which the laser beam may be focused such that the laser beam focus comes to rest on the surface of the workpiece 5. Furthermore, the laser beam aligning unit 10 may comprise a horizontal section for scanning the laser beam, by which the laser beam 1 can be scanned over a predefined area (e.g., to treat a surface of the workpiece 5). FIG. 1 illustrates the scanning area of the laser beam 1 in a side view and a scanning area comprising a rectangular and/or square shape on the surface of the workpiece 5, for example.

The vertical section of the laser beam aligning unit 10 may comprise an outer sleeve 3 and an inner sleeve 4. The inner sleeve 4 may be arranged inside the outer sleeve 3 in such a manner that the laser beam 1 may be guided through an inner chamber in a direction toward an area to be treated on workpiece 5.

The outer sleeve 3 and the inner sleeve 4 may comprise any arbitrary geometric shape. In one embodiment, respective sleeves may be implemented as cylindrically symmetrical around a beam axis of the laser beam 1 and comprise a continuously decreasing cross-section in the beam direction. The inner sleeve 4 may be attached centrally on a screen 6, connected to the inner wall of the outer sleeve 3 or an inner wall located above the upper section of the aligning unit 10, for example. The inner sleeve 4 may be integrally implemented with the screen 6 and may be manufactured from metal, for example. The light-outlet-side end of the outer sleeve 3 may protrude beyond that of the inner sleeve 4 by a predefined distance, for example. The light-outlet-side end of the outer sleeve 3 may be implemented such that the aligning unit 10 may be supported using the light-outlet-side end of the outer sleeve 3 on the workpiece 5 (e.g., for a tooth treatment, on the tooth to be treated and/or a surrounding area thereof). The light-outlet-side end of the inner sleeve 4 may be spaced apart based on a predefined distance from the area to be treated (e.g., associated with the surface of the workpiece 5). The front side of the inner sleeve 4 may be formed by a pane 7, transparent to the laser radiation (e.g., manufactured from diamond glass), for example.

One advantage provided by the arrangement illustrated in FIG. 1 is the laser beam 1 may be guided in the beam path to the surface to be treated inside the inner sleeve 4, and thus be protected from surroundings, for example. Accordingly, interfering absorption and/or scattering of the laser beam near the beam path may be mitigated (e.g., on ablated material particles), for example. Therefore, it may be desirable to implement the inner sleeve 4, so that the laser beam 1 merely exits from the inner sleeve 4 shortly above (e.g., as long as possible) the surface of the workpiece 5 to be treated. It may be appreciated that transparent pane 7 may be close to a focus of the laser beam 1, and at risk for damage at least because of existing residual absorption and/or multiphoton absorption of material of the transparent pane 7 at the wavelength of the laser beam 1. Therefore, in one embodiment, a space of a Rayleigh length (e.g., or more) may be maintained between the transparent disk 7 and the surface of the workpiece 5 to be treated, for example. The following equation applies for the Rayleigh length Z_{R}:

z_{R}=πw₀²/λ (1)

where w₀ is the radius of the beam cross-section in the focus of the laser beam. The following equation applies for the radius of the beam cross-section at the Rayleigh length z_{R}:

w(z_{R})=√2w₀ (2)

Thus, the intensity of the laser beam may be reduced to approximately 50% at the Rayleigh length z_{R}.

In one embodiment, the spacing between the transparent pane 7 and the light-outlet-side end of the outer sleeve 3 may comprise a length less than the Rayleigh length z_{R}. For example, if the residual absorption and/or the multiphoton absorption of the transparent pane 7 is determined to be acritical at the wavelength of the laser beam 1 (e.g., and thus does not result in destruction of the transparent pane 7), a spacing less than z_{R} may be selected, for example.

In one embodiment, selection of the spacing between the transparent pane 7 and the edge of the outer sleeve 3 may be selected based on a danger level associated with soiling the transparent pane 7 with ablated material. For example, ablated material may adhere to the transparent pane 7 (e.g., and thus contribute to increasing the absorption of the laser beam). That is, for example, it may be advisable to select a greater spacing between the transparent pane 7 and the light-outlet-side end of the outer sleeve 3 at least because of ablated material buildup on the transparent pane. In order to effectively counteract the danger of soiling of the transparent pane 7, transparent pane 7 may be cleaned in situ (e.g., in position), as will be described in greater detail below.

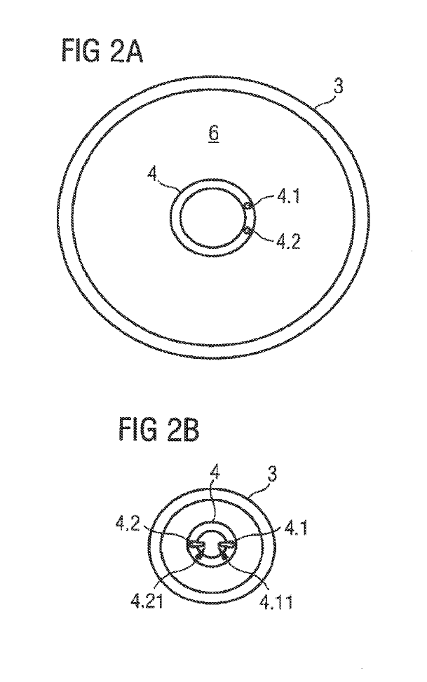

In one embodiment, the laser beam aligning unit 10 may supply liquid, gaseous, and/or plasma-like media to a location of a treatment on a surface of a workpiece 5 via supply lines, as identified by reference numerals 8 and 9, for example. The supply lines 8 and 9 may be guided through openings in the screen 6 and situated on their ends with suitable nozzles, for example. In one embodiment, supply lines 8 and 9 may be connected to channels 4.1 and 4.2, and integrated in a wall of inner sleeve 4. For example, channels 4.1 and 4.2 may be viewed as cross-sectional views in FIGS. 2A and 2B (e.g., at an upper end, in the starting area of the inner sleeve 4 (A) and at an end point, at the light-outlet-side end of the inner sleeve 4 (B)). Channels 4.1 and 4.2 may be designed along a conical jacket of the inner sleeve 4 as linearly extending channels comprising a constant angular position viewed in cross-section or as a curve comprising a variable angular position. At respective endpoints, channels 4.1 and 4.2 may be connected to nozzles 4.11 and 4.21 (e.g., adjustable nozzles configured to be controlled and/or aligned), for example.

Various media may, for example, be supplied to a treatment location on a surface of a workpiece 5 via supply lines 8 and 9 and/or channels 4.1 and 4.2. In one embodiment a cleaning agent, (e.g., such as water), may be supplied via a supply line, a channel connected thereto, and/or sprayed via a nozzle connected to the channel over the surface of the transparent pane 7 (e.g., to clean the transparent pane 7 of ablation particles deposited thereon), for example. Cleaning may be performed in situ (e.g., during ablation), and it may be ensured that the cleaning medium does not absorb laser radiation (e.g., if infrared laser radiation is used, no significant and/or noticeable absorption may occur in the infrared spectral range). FIG. 2B is an illustration of how a cleaning medium (e.g., such as water) is sprayed by nozzle 4.21 onto the outer surface of the transparent pane 7 such that transparent pane 7 may be cleaned of ablation particles, for example. The nozzle 4.21 may be aligned (e.g., permanently) inward, toward the surface of the transparent pane 7. In one embodiment, remaining nozzles (e.g., nozzle 4.11) connected to corresponding channels may be aligned on a point to be treated on the surface of the workpiece 5, for example.

As illustrated in the exemplary embodiment of FIG. 1, the outer sleeve 3 may also be implemented such that the outer sleeve may comprise a double wall structure, forming a chamber between the two walls implemented as a suction line. Outer sleeve 3 may be attached to a line extending to the upper section of the aligning unit connected to a pump, for example. As illustrated in FIGS. 2A and 2B, chamber 11 may comprise a cylindrically symmetrical shape, and may be aligned at a lower end in a direction toward a treatment location on a surface of the workpiece 5. In this way, ablated particles may be suctioned from one or more directions into chamber 11, for example. In the upper area, the chamber 11 may be connected to a line 12, for example.

According to one aspect, substances (e.g., such as organic or inorganic compositions, pharmaceutical substances, and/or air or water) may be supplied via supply lines 8 and 9 and/or channels 4.1 and 4.2. That is, for example, photosensitizers, plasmas, or nanoparticles as so-called sources of free electrons, so-called seed electrons, and/or directly exogenously generated free electrons may be supplied (e.g., exactly) to a focus point associated with a location of the treatment. In one embodiment, substances associated with improved protection of surrounding tissue (e.g., during tooth treatment) may be supplied as a pharmaceutical substance, to protect the surrounding tissue from thermal and/or chemical stress associated with tissue treatment, for example. To this end, ectoine and derivatives thereof, (e.g., an isotonic ectoine solution) may be used, and/or solutions comprising a same osmotic pressure as human blood (e.g., and thus mitigate bleeding, for example). In one embodiment, an isotonic ectoine solution may be provided, such that NaCl is added in a concentration range of 0.6%-0.7% to a 1% ectoine solution, for example.

In another embodiment, a partial vacuum and/or a vacuum may be generated between the outer sleeve 3 and the inner sleeve 4, for example. In this way, the lifetime (e.g., and therefore the free path length) of the injected free exogenous electrons may be increased, and (e.g., required light) power may be reduced, for example.

In the exemplary embodiment illustrated in FIGS. 1, 2A, and/or 2B, the inner sleeve 4 may comprise a rigid and/or integrally formed tube, for example. In one embodiment, inner sleeve 4 may comprise a telescope tube (e.g., multiple cylindrically tapering tubes and/or cylinders guided in a parallel fashion, which may be pulled out linearly up to a stop extension length (e.g., maximum length) and/or collapsed back inside one another), for example. In one embodiment, the length of the inner sleeve 4 may automatically vary such that a spacing between the workpiece surface and the protective glass 7 and/or the lower end of the inner sleeve 4 may be constant. In one embodiment, the spacing may correspond to a Rayleigh length, for example. Spacing may be based on a distance to protect protective glass in a manner such that the glass may be located outside of a focus of the laser beam, for example. The automatic variation of the length of the inner sleeve 4 may be associated with the automatic focusing of the laser beam 50 by an autofocus unit 2. In one embodiment, variation of the focal width caused by the autofocus unit 2 may cause an axial displacement of the laser focus, such that the laser focuses on the workpiece surface. The length of the inner sleeve 4 may be similarly varied, such that the spacing between the protective glass 7 and/or the surface or the laser focus remains approximately equal, for example.

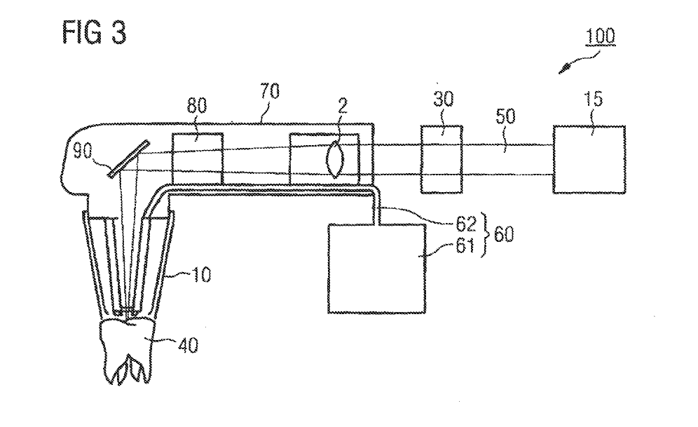

FIG. 3 illustrates an embodiment of a dental laser treatment device, comprising size ratios not drawn to scale. The laser treatment device 100 may comprise a laser beam source 15, configured to emit a pulsed treatment laser beam 50. The treatment laser beam 50 may be adjusted and focused on a surface of a tooth 40 to be treated, for example. The treatment laser beam may be shaped by a beam shaping unit 30 such that the beam 50 comprises a rectangular and/or top-hat beam profile. The laser beam 50 may be coupled into a handpiece 70 comprising an autofocus unit 20, a scanning unit 80, a deflection unit 90, and/or an aligning unit 10. The laser beam 50 may be supplied inside the handpiece 70 to the autofocus unit 20 comprising a lens 2, by which the laser beam 50 may be focused on the tooth surface in such a manner that the surface remains in the focus of the laser beam 50. The laser beam 50 may be scanned over a specific surface area by the scanning unit 80. The laser beam 50 may be deflected by an optical deflection unit 90, (e.g., such as a mirror, a mirror system comprising multiple mirrors, and/or a deflection prism) in the direction toward the tooth surface, for example.

In one embodiment, laser beam source 15 may be configured to generate laser pulses such that the pulses comprise a pulse duration in a range between 100 fs and 100 ns, and an energy per pulse in a range greater than 1 nJ. The focusing of the laser beam 50 may be set such that the laser beam 50 may comprise a focus diameter in a range from 1 μm-100 μm on the surface of the tooth 40. Furthermore, the laser beam source 15 may be configured to emit the laser pulses at a repetition rate in a range from 1 Hz-10 MHz.

In one embodiment, laser treatment device 100 may comprise a laser beam aligning unit 10 (e.g., as described above in connection with FIGS. 1 and 2A, B), for example. In another embodiment, laser treatment device 100 may comprise a distribution device 60 for distributing an ectoine solution in a direction toward a surrounding area of the tooth 40 to be treated, for example. That is, for example, distribution device 60 may comprise, a storage chamber 61 for storing the ectoine solution and/or a supply line 62 connected to the storage chamber 61. The supply line 62 may be connected to one of the channels of a second inner sleeve of the aligning unit, and be applied by a nozzle onto surroundings of the tooth to be treated, for example.

It may be appreciated that in exemplary embodiment of FIG. 3, two aspects of the present disclosure may be combined, (e.g., an aligning unit and a distribution device for distributing ectoine solution). The laser treatment device 100 may comprise an aligning unit according to FIGS. 1, 2A, and/or 2B, but no distribution device for distributing ectoine solution. Conversely, the laser treatment device 100 may comprise a distribution device, but no aligning unit (the laser treatment device 100 may comprise another aligning unit), for example.

Furthermore, the laser treatment device 100 may comprise distribution devices for other media, for example. In addition to conventional media (e.g., such as air or water), these can be substances such as photosensitizers, plasmas, and/or other types of electron sources, supported by ablation, for example. A distribution device for free electrons can be implemented like a Braun cathode ray tube, for example.

What is claimed is:

1. A dental laser treatment device for treatment of a tooth material, comprising:
a laser beam source configured to provide a pulsed treatment laser beam, wherein pulses of the pulsed treatment laser beam comprise a pulse duration in a range between 100 fs and 100 ns, an energy per pulse greater than 1 nJ, and a repetition rate in a range between 1 Hz and 10 MHz; and
a handpiece arranged in such a way that the pulsed treatment laser beam is coupled into the handpiece, the handpiece comprising a laser beam aligning unit, wherein:
the laser beam aligning unit comprises:
an outer sleeve with an open end configured to abut the tooth material to be treated, the outer sleeve comprising an outer wall and an inner wall and a chamber situated between the outer wall and the inner wall, the chamber being connectable to a pump and thus being configured as a suction line, and an open end of the chamber directed toward a treatment location of the tooth material to be treated; and
an inner sleeve arranged in such a way that the inner sleeve is surrounded by the outer sleeve in a spaced relationship to the inner wall of the outer sleeve, the inner sleeve having a light outlet end recessed from the open end of the outer sleeve such that the light outlet end of the inner sleeve is spaced above the tooth material to be treated, with the light outlet end of the inner sleeve being sealed by a non-focusing transparent pane, the inner sleeve comprising an inner chamber configured to guide the pulsed treatment laser beam through the inner sleeve and the non-focusing transparent pane in a direction toward the treatment location of the tooth material to be treated, wherein:
a distance by which the light outlet end of the inner sleeve is spaced above the tooth material to be treated is given by a Rayleigh length in relation to a focus of the pulsed treatment laser beam exiting from the light outlet end of the inner sleeve, and
the inner sleeve further comprises at least one supply line integrated in a wall thereof, the supply line being configured to supply liquid, gaseous or plasma-like media to the treatment location.

2. The dental laser treatment device of claim 1, comprising:
a scanning unit configured to scan an area using the laser beam source; and
a guide optic for the laser beam source configured to scan an area inside the inner sleeve.

3. The dental laser treatment device of claim 1, comprising a distribution device configured to distribute an ectoine solution.

4. The dental laser treatment device of claim 1, comprising a generation device configured to supply free electrons.

5. The dental laser treatment device of claim 1, wherein the supply line is connected at an end thereof to an adjustable nozzle configured to be controlled.

6. The dental laser treatment device of claim 5, wherein the adjustable nozzle is configured to be controlled in such a way that the adjustable nozzle is directed onto the treatment location.

7. The dental laser treatment device of claim 5, wherein the adjustable nozzle is configured to be controlled in such a way that the adjustable nozzle is directed onto the non-focusing transparent pane.
